(19) United States
(12) Patent Application Publication
(10) Pub. No.: US 2024/0370540 A1
(43) Pub. Date: Nov. 7, 2024

(54) IMAGE PROCESSING APPARATUS

(71) Applicant: KYOCERA Document Solutions Inc., Osaka-shi, Osaka (JP)

(72) Inventor: Shun TSUBOI, Osaka (JP)

(21) Appl. No.: 18/249,352

(22) PCT Filed: Oct. 20, 2022
(86) PCT No.: PCT/JP2022/039066

§ 371 Date: Apr. 17, 2023

(30) Foreign Application Priority Data
Oct. 25, 2021 (JP) 2021-173628

(51) Int. Cl. G06F 21/32

(52) U.S. Cl. CPC G06F 21/32

(57) ABSTRACT

The present invention aims at providing an image processing apparatus capable of contributing to daily health management of each user. An image processing apparatus (10) includes a user identification portion (6, 5c), a biometric portion (7), an information recording portion (54, S105), and an information output portion (54, S106). The biometric portion (7) measures biological information of a user who is present in front of the image forming apparatus (10). The information recording portion (54, S106) accumulates and records, every time a user identification is performed by the user identification portion (6, 5c), the biological information measured by the biometric portion (7) in a nonvolatile storage device (53) in association with user identification information. The information output portion (54, S105) is capable of outputting the biological information for each piece of the user identification information in a graph.

### TECHNICAL FIELD

The present invention relates to an image processing apparatus capable of managing a health condition of a user.

### BACKGROUND

An image processing apparatus such as a copying machine and a multifunction peripheral is sometimes shared by a plurality of users at a workplace and the like.

In addition, the image processing apparatus is known to have a function of identifying a user using face authentication (see, for example, Patent Literature 1).

### CITATION LIST

### Patent Literature

Patent Literature 1: Japanese Patent Application Publication No. 2016-148895

### SUMMARY

### Technical Problem

Incidentally, health management of employees in companies is becoming increasingly important. The image processing apparatus shared by a plurality of users may be able to contribute to daily health management of each user.

The present invention aims at providing an image processing apparatus capable of contributing to daily health management of each user.

### Solution to Problem

An image processing apparatus according to an embodiment of the present invention executes image processing requested by a user. The image processing apparatus includes a user identification portion, a biometric portion, an information recording portion, and an information output portion. The user identification portion identifies a user who is present in front of the image forming apparatus. The biometric portion measures biological information of the user who is present in front of the image forming apparatus. The information recording portion accumulates and records, every time a user identification is performed by the user identification portion, the biological information measured by the biometric portion in a nonvolatile storage device in association with user identification information. The information output portion is capable of outputting the biological information for each piece of the user identification information in a graph.

### Advantageous Effects of Invention

According to the present invention, an image processing apparatus capable of contributing to the daily health management of each user can be provided.

### BRIEF DESCRIPTION OF THE DRAWINGS

FIG. 1 is a block diagram showing a configuration of an image processing apparatus according to an embodiment.

FIG. 2 is a flowchart showing exemplary procedures of login processing in the image processing apparatus according to the embodiment.

FIG. 3 is a flowchart showing exemplary procedures of measurement reminder processing in the image processing apparatus according to the embodiment.

FIG. 4 is a diagram showing an exemplary configuration of registration user data in the image processing apparatus according to the embodiment.

FIG. 5 is a diagram showing an exemplary configuration of measurement history data in the image processing apparatus according to the embodiment.

### DETAILED DESCRIPTION

Hereinafter, an embodiment of the present invention will be described with reference to the drawings. It is noted that the following embodiment is an example of embodying the present invention and does not limit the technical scope of the present invention.

An image processing apparatus 10 according to the embodiment is an apparatus capable of executing image processing such as print processing and image reading processing. The image processing apparatus 10 executes the image processing requested by a user.

The print processing is processing of forming an image on a sheet 91. The image reading processing is processing of reading an image from a document sheet 92.

The image processing apparatus 10 is capable of communicating with other devices such as a host device 8. The image processing apparatus 10 is capable of communicating with a plurality of host devices 8 via a network 80.

For example, the image processing apparatus 10 is a copying machine, a facsimile apparatus, a multifunction peripheral, or the like. The multifunction peripheral has a function of the copying machine and a function of the facsimile apparatus.

The host device 8 is capable of communicating with the image processing apparatus 10. For example, the host device 8 generates print data and transmits a print request to the image processing apparatus 10 together with the generated print data.

### Configuration of Image Processing Apparatus 10

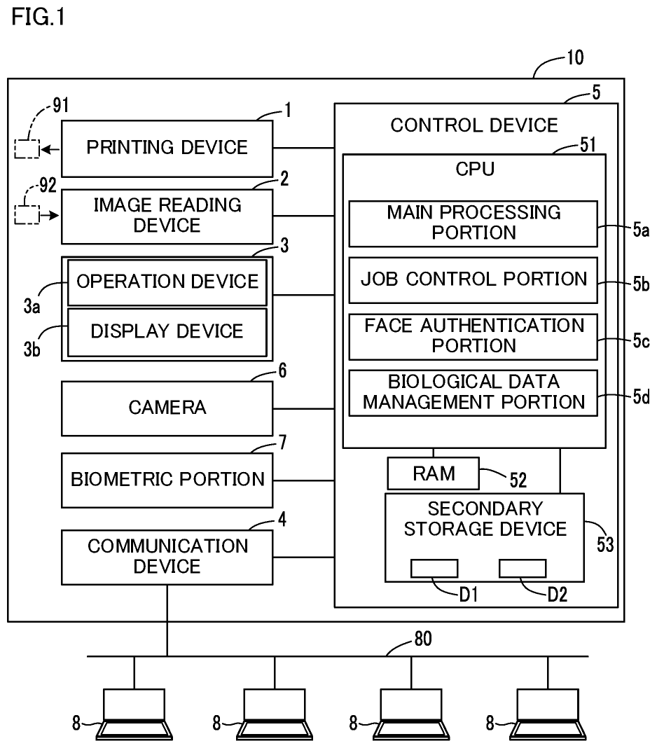

As shown in FIG. 1, the image processing apparatus 10 includes a printing device 1, an image reading device 2, a user interface device 3, a communication device 4, a control device 5, and the like. In addition, the image processing apparatus 10 includes a camera 6.

The printing device 1 executes the print processing using a predetermined system such as electrophotography or an ink-jet system. The printing device 1 is an example of a printing portion.

The image reading device 2 executes the image reading processing. The image reading device 2 includes a laser scanning portion which scans light on the document sheet 92 and an image sensor which receives reflected light that has been reflected by the document sheet 92.

The user interface device 3 includes an operation device 3a and a display device 3b. The operation device 3a is a device which accepts user operations. For example, the operation device 3a includes operation buttons, a touch panel, and the like. The display device 3b is capable of displaying information. For example, the display device 3b includes a display panel such as a liquid crystal panel.

The communication device 4 is a communication interface device which performs communication with other devices such as the host device 8 via the network 80.

The control device 5 performs all data transmissions and receptions with the other devices via the communication device 4.

In descriptions below, an image read from the document sheet 92 by the image reading processing of the image reading device 2 will be referred to as a read image.

The printing device 1 is capable of executing the print processing based on data of the read image or the print request received from the host device 8 via the communication device 4.

Further, the communication device 4 is capable of executing image data transmission processing for transmitting the data of the read image to a designated destination via the network 80.

The camera 6 captures an image of a face of a user who is present in front of the image processing apparatus 10.

The control device 5 executes various calculations, data processing, and control of various types of electric equipment included in the image processing apparatus 10. The control device 5 includes a CPU 51, a RAM (Random Access Memory) 52, a secondary storage device 53, and the like.

The secondary storage device 53 is a computer-readable nonvolatile storage device. The secondary storage device 53 is capable of storing computer programs and various types of data. For example, one or both of a hard disk drive and an SSD is/are adopted as the secondary storage device 53.

The secondary storage device 53 stores the computer programs to be executed by the CPU 51 and data to be referenced by the CPU 51. The CPU 51 is an example of a processor.

The CPU 51 is a processor that executes the computer programs stored in the secondary storage device 53 to execute the various types of data processing and control.

It is noted that other processors such as a DSP may execute the data processing and control in place of the CPU 51.

The RAM 52 is a computer-readable volatile storage device. The RAM 52 temporarily stores the computer programs to be executed by the CPU 51 and data to be output and referenced by the CPU 51 in a process of executing the computer programs.

The CPU 51 includes a plurality of processing modules that are realized by executing the computer programs. The plurality of processing modules in the CPU 51 include a main processing portion 5a, a job control portion 5b, a face authentication portion 5c, and the like.

The main processing portion 5a mainly monitors operations to the operation device 3a and data receptions by the communication device 4. In addition, when detecting an operation to the operation device 3a or a data reception, the main processing portion 5a controls a start of processing corresponding to the detected content.

The job control portion 5b controls the printing device 1, the image reading device 2, and the communication device 4. The job control portion 5b causes some or all of the printing device 1, the image reading device 2, and the communication device 4 to execute the requested image processing.

The printing device 1, the image reading device 2, and the communication device 4 are an example of an image processing portion capable of executing the image processing. In this embodiment, the image processing includes the print processing, the image reading processing, and the image data transmission processing.

The face authentication portion 5c executes face authentication processing that is based on an image acquired by the camera 6 to identify a user.

Registration user data D1 is stored in advance in the secondary storage device 53 (see FIG. 1). The registration user data D1 includes a plurality of pieces of individual user data D10 respectively corresponding to a plurality of users.

Each individual user data D10 includes a user code D11, registration authentication data D12, and destination data D13. The user code D11 is an identification code of a user. The user code D11 is an example of user identification information.

The registration authentication data D12 is pre-registered user authentication information. In this embodiment, the registration authentication data D12 is data that represents a feature amount in a face image of a user.

The face authentication portion 5c executes the face authentication processing that is based on the image acquired by the camera 6 to identify a user.

Specifically, the face authentication portion 5c specifies, as an input feature amount, a feature amount in a face of a user from an image acquired by the camera 6. In addition, the face authentication portion 5c collates the input feature amount with the registration authentication data D12. Further, the face authentication portion 5c specifies the user code D11 corresponding to the registration authentication data D12 that matches with the input feature amount.

The face authentication portion 5c being capable of specifying the registration authentication data D12 that matches with the input feature amount means that the face authentication processing has succeeded. The face authentication portion 5c being incapable of specifying the registration authentication data D12 that matches with the input feature amount means that the face authentication processing has failed.

When the face authentication processing has succeeded, the face authentication portion 5c allows the image processing apparatus 10 to execute the image processing. On the other hand, when the face authentication processing has failed, the face authentication portion 5c prohibits the image processing apparatus 10 from executing the image processing.

In this embodiment, the camera 6 and the face authentication portion 5c are an example of a user identification portion which identifies a user who is present in front of the image processing apparatus 10.

The destination data D13 is data that represents a pre-registered communication destination of each of the users. For example, the destination data D13 is an email address, an IP address, or the like.

Incidentally, health management of employees in companies is becoming increasingly important. The image processing apparatus 10 shared by the plurality of users may be able to contribute to daily health management of each user.

In this embodiment, the image processing apparatus 10 further includes a biometric portion 7. Further, the plurality of processing modules in the CPU 51 further include a biological data management portion 5d.

The biometric portion 7 measures biological information of a user who is present in front of the image processing apparatus 10. For example, the biological information includes one or both of a body temperature and pulse of a user.

For example, the biometric portion 7 includes an infrared sensor that measures a body temperature in a non-contact manner. In addition, the biometric portion 7 may also include a microwave Doppler sensor that measures a pulse in a non-contact manner.

For example, the face authentication portion 5c starts login processing when an authentication start operation with respect to the operation device 3a is detected. Alternatively, the face authentication portion 5c may start the login processing when a person is detected by the infrared sensor of the biometric portion 7.

In this embodiment, the login processing includes processing of causing the biometric portion 7 to measure the biological information and processing that uses the measurement result.

### Login Processing

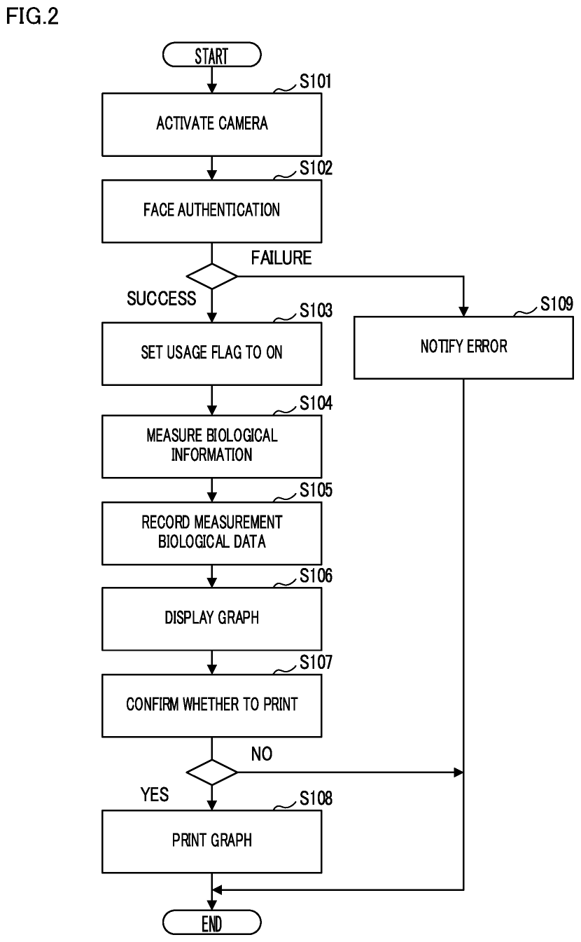

Hereinafter, exemplary procedures of the login processing will be described with reference to the flowchart shown in FIG. 2.

In descriptions below, S101, S102, . . . represent identification symbols of a plurality of steps in the login processing. In the login processing, processing of Step S101 is executed first.

### Step S101

In Step S101, the face authentication portion 5c activates the camera 6 and acquires data of a captured image from the camera 6.

After executing the processing of Step S101, the face authentication portion 5c shifts the processing to Step S102.

### Step S102

In Step S102, the face authentication portion 5c executes the face authentication processing. When the face authentication processing has succeeded, the face authentication portion 5c shifts the processing to Step S103. On the other hand, when the face authentication processing has succeeded, the face authentication portion 5c shifts the processing to Step S109.

Hereinafter, the user code D11 corresponding to the registration authentication data D12 that matches with the input feature amount in the face authentication processing will be referred to as a target user code.

### Step S103

In Step S103, the face authentication portion 5c sets a usage flag to ON. The usage flag is a flag that represents which of allowed or prohibited the execution of the image processing by the image processing apparatus 10 is. The usage flag is OFF in an initial state.

When the usage flag is ON, the job control portion 5b causes the image processing portion such as the printing device 1 or the image reading device 2 to execute the print processing corresponding to the request. On the other hand, when the usage flag is OFF, the job control portion 5b prohibits execution of the print processing corresponding to the request.

After executing the processing of Step S103, the face authentication portion 5c shifts the processing to Step S104.

### Step S104

In Step S104, the biological data management portion 5d activates the biometric portion 7. Thus, the biometric portion 7 measures the biological information of the user who is present in front of the image processing apparatus 10.

Hereinafter, data representing the biological information measured by the biometric portion 7 will be referred to as measurement biological data D22 (see FIG. 5). After executing the processing of Step S103, the biological data management portion 5d shifts the processing to Step S105.

### Step S105

In Step S105, the biological data management portion 5d records the measurement biological data D22 in the secondary storage device 53 in association with the target user code (see FIG. 1 and FIG. 5).

The processing of Step S105 is executed every time the face authentication processing succeeds. In other words, every time the face authentication processing succeeds, the biological data management portion 5d accumulates and records the measurement biological data D22 in the secondary storage device 53 in association with the target user code.

In this embodiment, every time the face authentication processing succeeds, the biological data management portion 5d records measurement date/time data D21 and the measurement biological data D22 in the secondary storage device 53 in association with the target user code. The measurement date/time data D21 represents a date and time on/at which the measurement biological data D22 has been acquired.

By executing the processing of Step S105, measurement history data D2 is accumulated in the secondary storage device 53 (see FIG. 5). The measurement history data D2 includes a plurality of pieces of individual history data D20 respectively corresponding to the plurality of users. Each individual history data D20 is associated with the user code D11. Each individual history data D20 includes the measurement date/time data D21 and the measurement biological data D22.

The biological data management portion 5d that executes the processing of Step S105 is an example of an information recording portion. After executing the processing of Step S104, the biological data management portion 5d shifts the processing to Step S106.

### Step S106

In Step S106, the biological data management portion 5d causes the display device 3b to display the measurement biological data D22 corresponding to the target user code in a graph.

By executing the processing of Step S106, the user can easily check his/her own biological information.

After executing the processing of Step S106, the biological data management portion 5d shifts the processing to Step S107.

### Step S107

In Step S107, the biological data management portion 5d executes print confirmation processing. The print confirmation processing is processing of confirming the user whether or not to print the graph of the measurement biological data D22.

When an operation to the operation device 3a that instructs to print the graph of the measurement biological data D22 is detected, the biological data management portion 5d shifts the processing to Step S108.

On the other hand, when an operation to the operation device 3a that instructs not to print the graph of the measurement biological data D22 is detected, the biological data management portion 5d ends the login processing.

### Step S108

In Step S108, the biological data management portion 5d causes the printing device 1 to execute processing of printing the graph of the measurement biological data D22 corresponding to the target user code.

In Step S106 or Step S109, the biological data management portion 5d is capable of outputting the measurement biological data D22 for each user code D11 in a graph. In addition, in Step S109, the biological data management portion 5d is capable of causing the printing device 1 to execute processing of forming an image that shows the measurement biological data D22 for each user code D11 in a graph, on the sheet 91. The biological data management portion 5d that executes the processing of Step S106 or Step S109 is an example of an information output portion.

After executing the processing of Step S108, the biological data management portion 5d ends the login processing.

### Step S109

In Step S109, the biological data management portion 5d executes error notification processing. The error notification processing is processing of outputting a notification that notifies that the face authentication processing has failed.

For example, in the error notification processing, the biological data management portion 5d causes the display device 3b to display an error message. After executing the processing of Step S109, the biological data management portion 5d ends the login processing.

By executing the processing of Step S104 to Step S108, the measurement biological data D22 for each user is accumulated by the plurality of users merely performing daily activities while using the image processing apparatus 10 at a workplace.

Furthermore, the plurality of users can easily check the measurement biological data D22 shown in a graph for each user while performing daily activities at the workplace. Thus, a mind of each user on health management is raised.

As described above, the image processing apparatus 10 can contribute to the daily health management of each user.

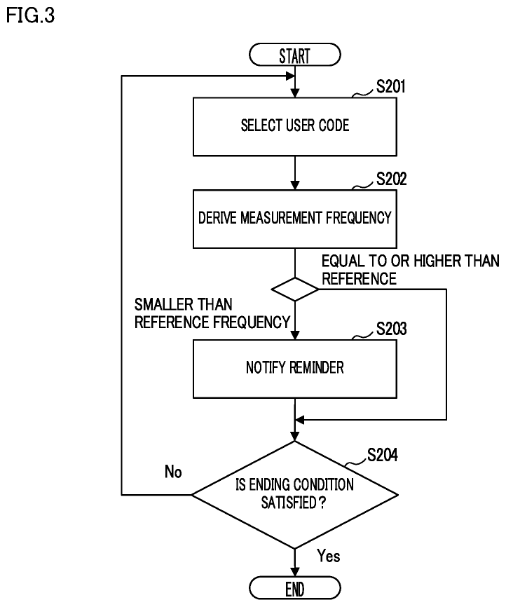

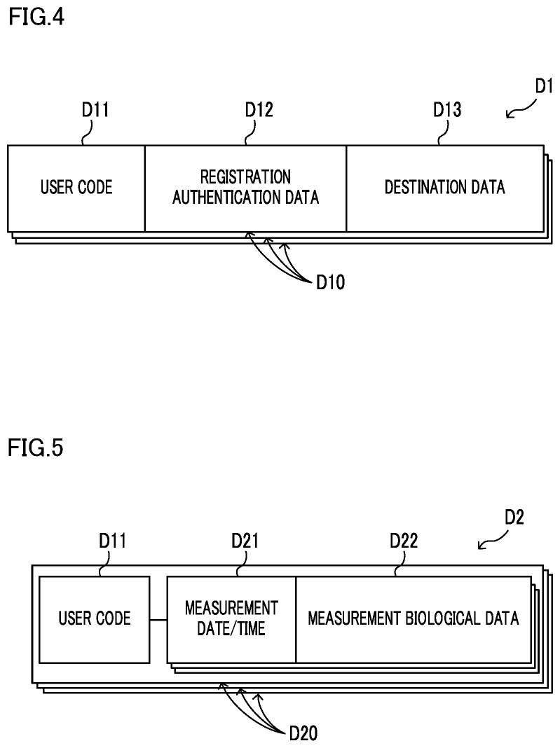

Even when the login processing is executed, the measurement biological data D22 is not sufficiently accumulated for a user whose usage frequency of the image processing apparatus 10 is low. In this regard, the biological data management portion 5d periodically executes measurement reminder processing (see FIG. 3).

### Measurement Reminder Processing

Hereinafter, exemplary procedures of the measurement reminder processing will be described with reference to the flowchart shown in FIG. 3.

In descriptions below, S201, S202, . . . represent identification symbols of a plurality of steps in the measurement reminder processing. In the measurement reminder processing, processing of Step S201 is executed first.

### Step S201

In Step S201, the biological data management portion 5d selects one of the plurality of user codes D11 in the registration user data D1.

In descriptions below, the selected user code D11 will be referred to as a selection user code. After executing the processing of Step S201, the biological data management portion 5d shifts the processing to Step S202.

### Step S202

In Step S202, the biological data management portion 5d derives a measurement frequency corresponding to the selection user code. The measurement frequency is a recording frequency of the measurement biological data D22 corresponding to the selection user code.

In this embodiment, the biological data management portion 5d derives the measurement frequency based on the measurement date/time data D21 corresponding to the selection user code.

In other words, in Step S202, the biological data management portion 5d determines, for each user code D11, whether or not the measurement frequency falls below the preset reference frequency. The biological data management portion 5d that executes the processing of Step S202 is an example of a frequency determination portion.

When the measurement frequency falls below the preset reference frequency, the biological data management portion 5d shifts the processing to Step S203. On the other hand, when the measurement frequency is equal to or higher than the reference frequency, the biological data management portion 5d shifts the processing to Step S204.

### Step S203

In Step S203, the biological data management portion 5d executes reminder notification processing. The biological data management portion 5d that executes the reminder notification processing is an example of a notification portion.

The reminder notification processing includes processing of specifying the destination data D13 corresponding to the selection user code. In addition, the reminder notification processing includes processing of transmitting a reminder notification to a destination indicated by the specified destination data D13.

The reminder notification is a notification that prompts the user to log in to the image processing apparatus 10. In this embodiment, logging in to the image processing apparatus 10 means measuring the biological information in the image processing apparatus 10.

The destination data D13 specified in Step S203 is data of a target destination. The target destination data is data associated with the user code D11 corresponding to the measurement biological data D22, the measurement frequency of which has been determined as falling below the reference frequency, out of the plurality of pieces of pre-registered destination data D13.

After executing the processing of Step S203, the biological data management portion 5d shifts the processing to Step S204.

### Step S204

In Step S204, the biological data management portion 5d determines whether or not a predetermined ending condition is satisfied.

For example, the ending condition is a condition that the processing of Step S202 to Step S203 has been executed for all of the plurality of user codes D11 in the registration user data D1.

When determining that the ending condition is not satisfied, the biological data management portion 5d shifts the processing to Step S201. On the other hand, when determining that the ending condition is satisfied, the biological data management portion 5d ends the measurement reminder processing.

By periodically executing the measurement reminder processing, accumulation of the measurement biological data D22 is facilitated even for a user whose usage frequency of the image processing apparatus 10 is low.

1.-6. (canceled)

7. An image processing apparatus which executes image processing requested by a user, comprising:
a user identification portion which identifies a user who is present in front of the image forming apparatus;
a biometric portion which measures biological information of the user who is present in front of the image forming apparatus;
an information recording portion which accumulates and records, every time a user identification is performed by the user identification portion, the biological information measured by the biometric portion in a nonvolatile storage device in association with user identification information;
an information output portion capable of outputting the biological information for each piece of the user identification information in a graph;
a frequency determination portion which determines, for each piece of the user identification information, whether or not a recording frequency of the biological information falls below a preset reference frequency; and
a notification portion which specifies, out of a plurality of preregistered communication destinations, a target destination associated with the user identification information corresponding to the biological information whose recording frequency has been determined as falling below the reference frequency, and executes notification processing with respect to the target destination.

8. The image processing apparatus according to claim 7, wherein
the user identification portion includes
a camera which captures an image of a face of the user, and
a face authentication portion which executes face authentication processing that is based on the image captured by the camera to identify the user.

9. The image processing apparatus according to claim 7, wherein
the biometric portion measures the biological information in a non-contact manner.

10. The image processing apparatus according to claim 7, wherein
the biological information includes one or both of a body temperature and pulse of the user.

11. The image processing apparatus according to claim 7, comprising:
a printing portion which forms an image on a sheet,
wherein the information output portion is capable of causing the printing portion to execute processing of forming, on the sheet, an image that shows the biological information for each piece of the user identification information in a graph.
